(19) United States
(12) Patent Application Publication
(10) Pub. No.: US 2021/0005328 A1
(43) Pub. Date: Jan. 7, 2021

(54) Expectedness Cognitive Service for Pharmacovigilence

(71) Applicant: International Business Machines Corporation, Armonk, NY (US)

(72) Inventors: Abhinandan Kelgere Ramesh, San Jose, CA (US); Ramani Routray, San Jose, CA (US); Claire Abu-Assal, Pismo Beach, CA (US); Sheng Hua Bao, San Jose, CA (US)

(21) Appl. No.: 16/458,450

(22) Filed: Jul. 1, 2019

(51) Int. Cl. G16H 70/40; G06N 3/08; G16H 10/60; G16H 50/30; G06N 20/00

(52) U.S. Cl. CPC G16H 70/40; G06N 3/08; G06N 3/04; G16H 50/30; G06N 20/00; G16H 10/60

(57) ABSTRACT

A mechanism is provided in a data processing system comprising a processor and a memory, the memory comprising instructions that are executed by the processor to specifically configure the processor to implement an expectedness cognitive service for identifying seriousness of a patient case. The expectedness cognitive service receives a patient case and identifies a suspect drug, an adverse event, and context features based on the patient case. An expectedness binary classifier within the expectedness cognitive service determines a plurality of expectedness classifications for the adverse event with respect to a plurality of drug labeling service repositories. The expectedness cognitive service generates and outputs an expectedness classification output comprising the plurality of expectedness classifications.

### BACKGROUND

The present application relates generally to an improved data processing apparatus and method and more specifically to mechanisms for expectedness cognitive service for pharmacovigilence.

An electronic health record (EHR) or electronic medical record (EMR) is the systematized collection of patient and population electronically-stored health information in a digital format. These records can be shared across different health care settings. Records are shared through network-connected, enterprise-wide information systems or other information networks and exchanges. EMRs may include a range of data, including demographics, medical history, medication and allergies, immunization status, laboratory test results, radiology images, vital signs, personal statistics like age and weight, and billing information.

EMR systems are designed to store data accurately and to capture the state of a patient across time. It eliminates the need to track down a patient's previous paper medical records and assists in ensuring data is accurate and legible. It can reduce risk of data replication as there is only one modifiable file, which means the file is more likely up to date, and decreases risk of lost paperwork. Due to the digital information being searchable and in a single file, EMRs are more effective when extracting medical data for the examination of possible trends and long term changes in a patient. Population-based studies of medical records may also be facilitated by the widespread adoption of EMRs.

Health Level Seven International (HL7) is a not-for-profit, American National Standards Institute (ANSI) accredited standards developing organization dedicated to providing a comprehensive framework and related standards for the exchange, integration, sharing, and retrieval of electronic health information that supports clinical practice and the management, delivery and evaluation of health services. The HL7 Individual Case Safety Report (ICSR) captures information needed to support reporting of adverse events, product problems and consumer complaints associated with the use of U.S. Food and Drug Administration (FDA) regulated products. The FDA Adverse Event Reporting System (FAERS) is a database that contains adverse event reports, medication error reports and product quality complaints resulting in adverse events that were submitted to FDA. The database is designed to support the FDA's post-marketing safety surveillance program for drug and therapeutic biologic products. FAERS is a useful tool for FDA for activities such as looking for new safety concerns that might be related to a marketed product, evaluating a manufacturer's compliance to reporting regulations and responding to outside requests for information. The reports in FAERS are evaluated by clinical reviewers, in the Center for Drug Evaluation and Research (CDER) and the Center for Biologics Evaluation and Research (CBER), to monitor the safety of products after they are approved by FDA.

Healthcare professionals, consumers, and manufacturers submit reports to FAERS. FDA receives voluntary reports directly from healthcare professionals (such as physicians, pharmacists, nurses and others) and consumers (such as patients, family members, lawyers and others). Healthcare professionals and consumers may also report to the products' manufacturers. If a manufacturer receives a report from a healthcare professional or consumer, it is required to send the report to FDA as specified by regulations.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided in a data processing system comprising a processor and a memory, the memory comprising instructions that are executed by the processor to specifically configure the processor to implement an expectedness cognitive service for identifying seriousness of a patient case. The method comprises receiving, by the expectedness cognitive service executing in the data processing system, a patient case. The method further comprises identifying, by the expectedness cognitive service, a suspect drug, an adverse event, and context features based on the patient case. The method further comprises determining, by an expectedness binary classifier within the expectedness cognitive service, a plurality of expectedness classifications for the adverse event with respect to a plurality of drug labeling service repositories. The method further comprises generating and outputting, by the expectedness cognitive service, an expectedness classification output comprising the plurality of expectedness classifications.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive healthcare system in a computer network.

FIG. 2 is a block diagram of an example data processing system in which aspects of the illustrative embodiments are implemented;

FIG. 3 is an example diagram illustrating an interaction of elements of a healthcare cognitive system in accordance with one illustrative embodiment;

FIG. 4 is a block diagram of a seriousness cognitive service in accordance with an illustrative embodiment;

FIG. 5 depicts an example of model input and output for the seriousness cognitive service in accordance with an illustrative embodiment;

FIG. 6 is a block diagram illustrating a seriousness determination cognitive module for a seriousness cognitive service in accordance with an illustrative embodiment;

FIG. 7 depicts an example of model input and output for the expectedness cognitive service in accordance with an illustrative embodiment;

FIG. 8 is a block diagram illustrating an expectedness determination cognitive module for an expectedness cognitive service in accordance with an illustrative embodiment;

FIG. 9 is a flowchart illustrating operation of a mechanism for training a seriousness cognitive service model in accordance with an illustrative embodiment;

FIG. 10 is a flowchart illustrating operation of a seriousness cognitive service in accordance with an illustrative embodiment;

FIG. 11 is a flowchart illustrating operation of a mechanism for training an expectedness cognitive service model in accordance with an illustrative embodiment; and

FIG. 12 is a flowchart illustrating operation of an expectedness cognitive service in accordance with an illustrative embodiment.

### DETAILED DESCRIPTION

FAERS data has limitations. First, there is no certainty that a reported event (adverse event or medication error) was due to the product. FDA does not require that a causal relationship between a product and event be proven, and reports do not always contain enough detail to properly evaluate an event. Furthermore, FDA does not receive reports for every adverse event or medication error that occurs with a product. Many factors can influence whether an event will be reported, such as the time a product has been marketed and publicity about an event.

In one illustrative embodiment, a seriousness cognitive service is provided that analyzes patient case information to identify instances of adverse events and categorizes these adverse events based on seriousness category. To generate these seriousness category results, the seriousness cognitive service may employ rules to evaluate seriousness features in the context of the adverse event to thereby generate a seriousness level of the adverse event (AE). Consolidation rules are provided for consolidating the seriousness determination for each AE associated with the patient to generate a case seriousness level. The case seriousness level may be used to generate notifications that may include a rationale for the case seriousness level as indicated by the individual AE seriousness level determinations, e.g., identifying sections of patient information that prove the rationale of the seriousness determination.

The seriousness cognitive service provides a cognitive evaluation of the patient information across all AEs to determine a case seriousness level determination, which may be reported along with rationale information. The seriousness cognitive service accurately identifies the seriousness of a patient's case in a timely manner to abide by reporting requirements and to provide quality care to the patient.

In one illustrative embodiment, an expectedness cognitive service is provided that evaluates the expectedness of an adverse event (AE) associated with a drug. The expectedness cognitive service evaluates a plurality of different conventions used to determine whether a particular AE is an expected side effect of a drug. These conventions may be due to different standards for specifying drug side effects based on countries, geographies, etc. The expectedness cognitive service determines for each combination of evaluations under the various conventions, what the expectedness is at a tuple granularity. The expectedness cognitive service looks at both a repository of drug label information and the like indicating expected side effects and the context of adverse events in the patient documentation to determine if the AE is expected. The expectedness cognitive service outputs an indication of whether the AE is an expected event or not.

The expectedness cognitive service provides a more accurate indication of expectedness of a side effect for a drug and looking at only a single drug label service repository through a manual process. The expectedness cognitive service automatically takes into consideration a cognitive evaluation of the context of adverse events when determining expectedness, which provides a more accurate result that is less prone to error due to human intervention.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, sofhvare executing on general purpose hardware, software instructions stored on a medium such that the instructions are readily executable by specialized or general purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine” or “service,” if used herein with regard to describing embodiments and features of the invention, is not intended to be limiting of any particular implementation for accomplishing and/or performing the actions, steps, processes, etc., attributable to and/or performed by the engine or service. An engine or service may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine or service is, unless otherwise specified, for purposes of convenience of reference and not intended to be limiting to a specific implementation. Additionally, any functionality attributed to an engine or service may be equally performed by multiple engines, incorporated into and/or combined with the functionality of another engine of the same or different type, or distributed across one or more engines or services of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

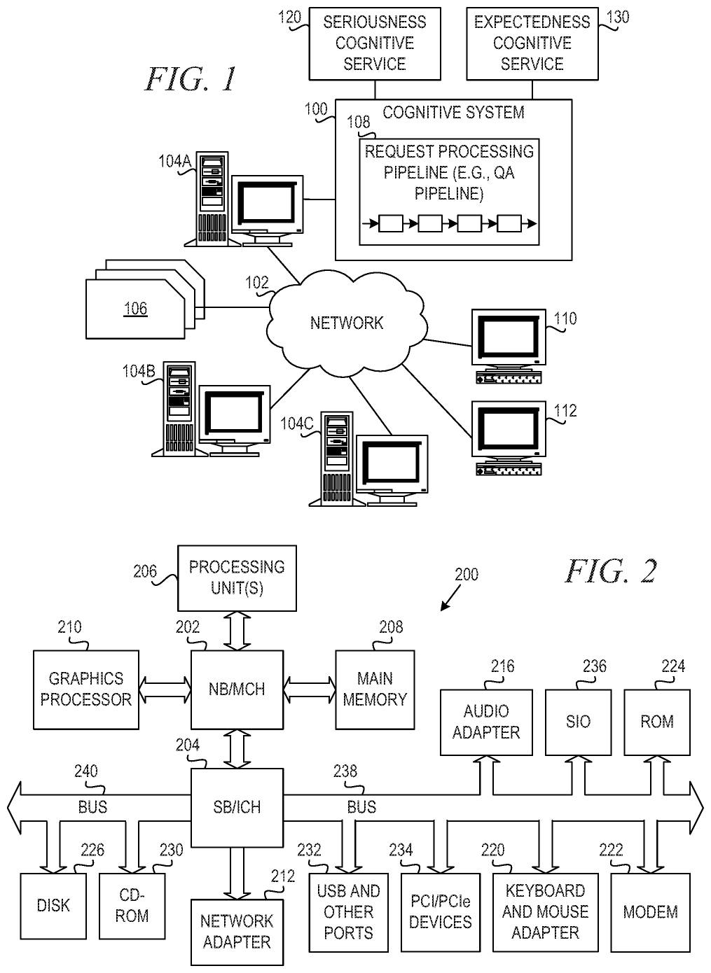

The illustrative embodiments may be utilized in many different types of data processing environments. In order to provide a context for the description of the specific elements and functionality of the illustrative embodiments, FIGS. 1-3 are provided hereafter as example environments in which aspects of the illustrative embodiments may be implemented. It should be appreciated that FIGS. 1-3 are only examples and are not intended to assert or imply any limitation with regard to the environments in which aspects or embodiments of the present invention may be implemented. Many modifications to the depicted environments may be made without departing from the spirit and scope of the present invention.

FIGS. 1-3 are directed to describing an example cognitive system for healthcare applications (also referred to herein as a “healthcare cognitive system”) which implements a request processing pipeline, request processing methodology, and request processing computer program product with which the mechanisms of the illustrative embodiments are implemented. These requests may be provided as structured or unstructured request messages or any other suitable format for requesting an operation to be performed by the healthcare cognitive system. As described in more detail hereafter, the particular healthcare application that is implemented in the cognitive system of the present invention is a healthcare application for cognitive analysis and disambiguation of electronic medical records for presentation of pertinent information for a medical treatment plan.

It should be appreciated that the healthcare cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests, depending on the desired implementation. For example, in some cases, a first request processing pipeline may be trained to operate on input requests directed to a first medical malady domain (e.g., various types of blood diseases) while another request processing pipeline may be trained to answer input requests in another medical malady domain (e.g., various types of cancers). In other cases, for example, the request processing pipelines may be configured to provide different types of cognitive functions or support different types of healthcare applications, such as one request processing pipeline being used for patient diagnosis, another request processing pipeline being configured for cognitive analysis of EMR data, another request processing pipeline being configured for patient monitoring, etc.

Moreover, each request processing pipeline may have its own associated corpus or corpora that it ingests and operate on, e.g., one corpus for blood disease domain documents and another corpus for cancer diagnostics domain related documents in the above examples. In some cases, the request processing pipelines may each operate on the same domain of input requests but may have different configurations, e.g., different annotators or differently trained annotators, such that different analysis and potential answers are generated. The healthcare cognitive system may provide additional logic for routing input requests to the appropriate request processing pipeline, such as based on a determined domain of the input request, combining and evaluating final results generated by the processing performed by multiple request processing pipelines, and other control and interaction logic that facilitates the utilization of multiple request processing pipelines.

As will be discussed in greater detail hereafter, the illustrative embodiments may be integrated in, augment, and extend the functionality of the request processing pipeline and mechanisms of a healthcare cognitive system with regard to an electronic medical record completeness and data quality assessment mechanism.

Thus, it is important to first have an understanding of how cognitive systems in a cognitive system implementing a request processing pipeline is implemented before describing how the mechanisms of the illustrative embodiments are integrated in and augment such cognitive systems and request processing pipeline mechanisms. It should be appreciated that the mechanisms described in FIGS. 1-3 are only examples and are not intended to state or imply any limitation with regard to the type of cognitive system mechanisms with which the illustrative embodiments are implemented. Many modifications to the example cognitive system shown in FIGS. 1-3 may be implemented in various embodiments of the present invention without departing from the spirit and scope of the present invention.

FIG. 1 depicts a schematic diagram of one illustrative embodiment of a cognitive system 100 implementing a request processing pipeline 108 in a computer network 102. The cognitive system 100 is implemented on one or more computing devices 104A-C (comprising one or more processors and one or more memories, and potentially any other computing device elements generally known in the art including buses, storage devices, communication interfaces, and the like) connected to the computer network 102. For purposes of illustration only, FIG. 1 depicts the cognitive system 100 being implemented on computing device 104A only, but as noted above the cognitive system 100 may be distributed across multiple computing devices, such as a plurality of computing devices 104A-C. The network 102 includes multiple computing devices 104A-C, which may operate as server computing devices, and 110-112 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 100 and network 102 may provide cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive system 100 may be used with components, systems, sub-systems, and/or devices other than those that are depicted herein.

The cognitive system 100 is configured to implement a request processing pipeline 108 that receive inputs from various sources. The requests may be posed in the form of a natural language request, natural language request for information, natural language request for the performance of a cognitive operation, or the like. For example, the cognitive system 100 receives input from the network 102, a corpus or corpora of electronic documents 106, cognitive system users, and/or other data and other possible sources of input. In one embodiment, some or all of the inputs to the cognitive system 100 are routed through the network 102. The various computing devices 104A-C on the network 102 include access points for content creators and cognitive system users. Some of the computing devices 104A-C include devices for a database storing the corpus or corpora of data 106 (which is shown as a separate entity in FIG. 1 for illustrative purposes only). Portions of the corpus or corpora of data 106 may also be provided on one or more other network attached storage devices, in one or more databases, or other computing devices not explicitly shown in FIG. 1. The network 102 includes local network connections and remote connections in various embodiments, such that the cognitive system 100 may operate in environments of any size, including local and global, e.g., the Internet.

In one embodiment, the content creator creates content in a document of the corpus or corpora of data 106 for use as part of a corpus of data with the cognitive system 100. The document includes any file, text, article, or source of data for use in the cognitive system 100. Cognitive system users access the cognitive system 100 via a network connection or an Internet connection to the network 102, and input requests to the cognitive system 100 that are processed based on the content in the corpus or corpora of data 106. In one embodiment, the requests are formed using natural language. The cognitive system 100 parses and interprets the request via a pipeline 108, and provides a response to the cognitive system user, e.g., cognitive system user 110, containing one or more response to the request, results of processing the request, or the like. In some embodiments, the cognitive system 100 provides a response to users in a ranked list of candidate responses while in other illustrative embodiments, the cognitive system 100 provides a single final response or a combination of a final response and ranked listing of other candidate responses.

The cognitive system 100 implements the pipeline 108 which comprises a plurality of stages for processing an input request based on information obtained from the corpus or corpora of data 106. The pipeline 108 generates responses for the input request based on the processing of the input request and the corpus or corpora of data 106.

As noted above, while the input to the cognitive system 100 from a client device may be posed in the form of a natural language request, the illustrative embodiments are not limited to such. Rather, the input request may in fact be formatted or structured as any suitable type of request which may be parsed and analyzed using structured and/or unstructured input analysis, including but not limited to the natural language parsing and analysis mechanisms of a cognitive system such as IBM Watson™, to determine the basis upon which to perform cognitive analysis and providing a result of the cognitive analysis. In the case of a healthcare based cognitive system, this analysis may involve processing patient medical records, medical guidance documentation from one or more corpora, and the like, to provide a healthcare oriented cognitive system result.

In the context of the present invention, cognitive system 100 may provide a cognitive functionality for assisting with healthcare based operations. For example, depending upon the particular implementation, the healthcare based operations may comprise patient diagnostics medical practice management systems, personal patient care plan generation and monitoring, or patient electronic medical record (EMR) evaluation for various purposes. Thus, the cognitive system 100 may be a healthcare cognitive system 100 that operates in the medical or healthcare type domains and which may process requests for such healthcare operations via the request processing pipeline 108 input as either structured or unstructured requests, natural language input, or the like. In the illustrative embodiment, the cognitive system 100 may be a drug safety cognitive system that performs operations for pharmacovigilance.

Pharmacovigilance, also known as drug safety, is the pharmacological science relating to the collection, detection, assessment, monitoring, and prevention of adverse effects with pharmaceutical products. Pharmacovigilance focuses on adverse drug reactions, which are defined as any response to a drug that is noxious and unintended, including lack of efficacy. Medication errors such as overdose, and misuse and abuse of a drug as well as drug exposure during pregnancy and breastfeeding, are also of interest, even without an adverse event, because they may result in an adverse drug reaction. Information received from patients and healthcare providers via pharmacovigilance agreements (PVAs), as well as other sources, such as the medical literature, plays a critical role in providing the data necessary for pharmacovigilance to take place. In fact, in order to market or to test a pharmaceutical product in most countries, adverse event data received by the license holder must be submitted to the local drug regulatory authority. Ultimately, pharmacovigilance is concerned with identifying the hazards associated with pharmaceutical products and with minimizing the risk of any harm that may come to patients. Companies must conduct a comprehensive drug safety and pharmacovigilance audit to assess their compliance with worldwide laws, regulations, and guidance.

As shown in FIG. 1, the cognitive system 100 is further augmented, in accordance with the mechanisms of the illustrative embodiments, to include logic implemented in specialized hardware, software executed on hardware, or any combination of specialized hardware and software executed on hardware, for implementing a seriousness cognitive service 120 that accurately identifies the seriousness of an adverse event in a timely manner to abide by reporting requirements and to provide quality of care to the patient.

In accordance with another illustrative embodiment, the cognitive system 100 is augmented to include logic implemented in a specialized hardware, software executed on hardware, or any combination of specialized hardware and software executed on hardware, for implementing an expectedness cognitive service 130 that evaluates the expectedness of an adverse event due to a drug taken by a patient.

In the illustrative embodiment, seriousness cognitive service 120 and expectedness cognitive service 130 are independent. Serious cognitive service 120 may exist without the expectedness cognitive service 130, and expectedness cognitive service 130 may exist without the seriousness cognitive service 120. In one example embodiment, expectedness cognitive service 130 may receive a seriousness value from seriousness cognitive service 120 as an input; however, in the alternative, expectedness cognitive service 130 may receive a seriousness value from another source, such as from a human expert.

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 2 is provided as an example of one type of data processing system in which aspects of the present invention may be implemented. Many other types of data processing systems may be likewise configured to specifically implement the mechanisms of the illustrative embodiments.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which, which implements a cognitive system 100 and QA system pipeline 108 augmented to include the additional mechanisms of the illustrative embodiments described hereafter.

In the depicted example, data processing system 200 employs a hub architecture including North Bridge and Memory Controller Hub (NB/MCH) 202 and South Bridge and Input/Output (IO) Controller Hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows 10®. An object-oriented programming system, such as the Java™ programming system, may run in conjunction with the operating system and provides calls to the operating system from Java programs or applications executing on data processing system 200.

As a server, data processing system 200 may be, for example, an IBM® eServer™ System p® computer system, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

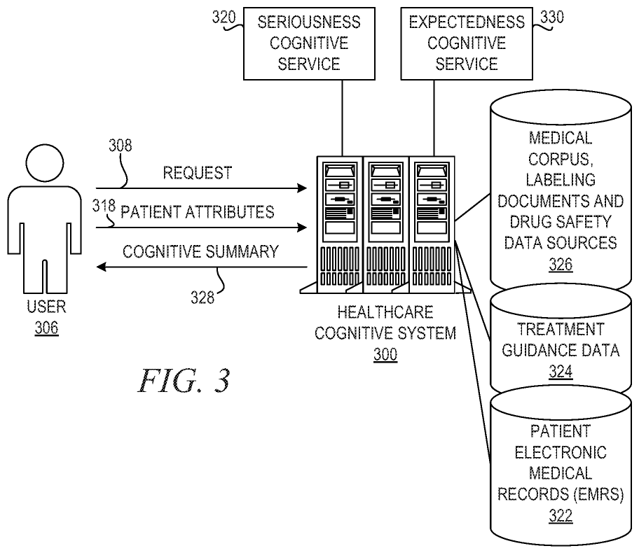

FIG. 3 is an example diagram illustrating an interaction of elements of a healthcare cognitive system in accordance with one illustrative embodiment. The example diagram of FIG. 3 depicts an implementation of a healthcare cognitive system 300 that is configured to provide a cognitive summary of EMR data for patients, to accurately identify the seriousness of an adverse event, and to evaluate the expectedness of an adverse event due to a drug taken by a patient. However, it should be appreciated that this is only an example implementation and other healthcare operations may be implemented in other embodiments of the healthcare cognitive system 300 without departing from the spirit and scope of the present invention.

Moreover, it should be appreciated that while FIG. 3 depicts the user 306 as a human figure, the interactions with user 306 may be performed using computing devices, medical equipment, and/or the like, such that user 306 may in fact be a computing device, e.g., a client computing device. For example, interactions between the user 306 and the healthcare cognitive system 300 will be electronic via a user computing device (not shown), such as a client computing device 110 or 112 in FIG. 1, communicating with the healthcare cognitive system 300 via one or more data communication links and potentially one or more data networks.

As shown in FIG. 3, in accordance with one illustrative embodiment, the user 306 submits a request 308 to the healthcare cognitive system 300, such as via a user interface on a client computing device that is configured to allow users to submit requests to the healthcare cognitive system 300 in a format that the healthcare cognitive system 300 can parse and process. The request 308 may include, or be accompanied with, information identifying patient attributes 318. These patient attributes 318 may include, for example, an identifier of the patient 302 from which patient EMRs 322 for the patient may be retrieved, demographic information about the patient, symptoms, and other pertinent information obtained from responses to requests or information obtained from medical equipment used to monitor or gather data about the condition of the patient. Any information about the patient that may be relevant to a cognitive evaluation of the patient by the healthcare cognitive system 300 may be included in the request 308 and/or patient attributes 318.

The healthcare cognitive system 300 provides a cognitive system that is specifically configured to perform an implementation specific healthcare oriented cognitive operation. In the depicted example, this healthcare oriented cognitive operation is directed to providing a cognitive summary of EMR data 328 to the user 306 to assist the user 306 in treating the patient based on their reported symptoms and other information gathered about the patient. The healthcare cognitive system 300 operates on the request 308 and patient attributes 318 utilizing information gathered from the medical corpus, labeling documents and drug safety data sources, and other source data 326, treatment guidance data 324, and the patient EMRs 322 associated with the patient to generate cognitive summary 328. The cognitive summary 328 may be presented in a ranked ordering with associated supporting evidence, obtained from the patient attributes 318 and data sources 322-326, indicating the reasoning as to why portions of EMR data 322 are being provided.

In accordance with the illustrative embodiments herein, the healthcare cognitive system 300 may be implemented as a drug safety system and is augmented to include a seriousness cognitive service 320 that accurately identifies the seriousness of an adverse event (AE). In another illustrative embodiment, the healthcare cognitive system 300 is augmented to include an expectedness cognitive service 330 that evaluates the expectedness of an AE due to a drug taken by a patient. In another embodiment, seriousness cognitive service 320 and expectedness cognitive service 330 to augment and improve the results of healthcare cognitive system 300. For example, healthcare cognitive service 300 may generate a cognitive summary 328 including one or more seriousness category results and an expectedness result. In another example embodiment, the expectedness result may depend on the one or more seriousness category results.

Seriousness cognitive service 320 analyzes patient case information to identify instances of adverse events and categorizes these adverse events to generate tuples [AdverseEvent, SeriousnessCategory]. To generate these adverse event tuples, rules may be employed to evaluate seriousness features in the context of the adverse event to thereby generate a seriousness level of the adverse event. Consolidation rules are provided for consolidating the seriousness determination for each adverse event associated with the patient to generate a case seriousness level. The case seriousness level may be used to generate notifications that may include a rationale for the case seriousness level as indicated by the individual adverse event seriousness level determinations, e.g., identifying sections of patient information that provide the rationale of the seriousness determination. Seriousness cognitive service 320 uses data sources 326, including drug safety data sources, such as spontaneous reports, clinical trials, medical literature, legal documents, social media/patient support programs, etc.

Expectedness cognitive service 330 evaluates the expectedness of an adverse event associated with a drug. The expectedness cognitive service 330 evaluates a plurality of different conventions used to determine whether a particular adverse event is an expected side effect of a drug. These conventions may be due to different standards for specifying drug side effects based on countries, geographies, etc. The expectedness cognitive service 330 determines for each combination of evaluations under the various conventions what the expectedness is at a tuple granularity. The expectedness cognitive service 330 looks at both a repository of drug label information and the like indicating expected side effects and the context of adverse events in the patient documentation to determine whether the adverse event is expected. The expectedness cognitive service 330 outputs an indication of whether the adverse event is an expected event or not. Expectedness cognitive service 330 uses data sources 326, including drug safety data sources and labeling documents, such as Investigator's Brochure (IB), Summary of Product Characteristics (SMPC), Company Core Data Sheet (CCDS), United States Prescribing Information (USPI), etc.

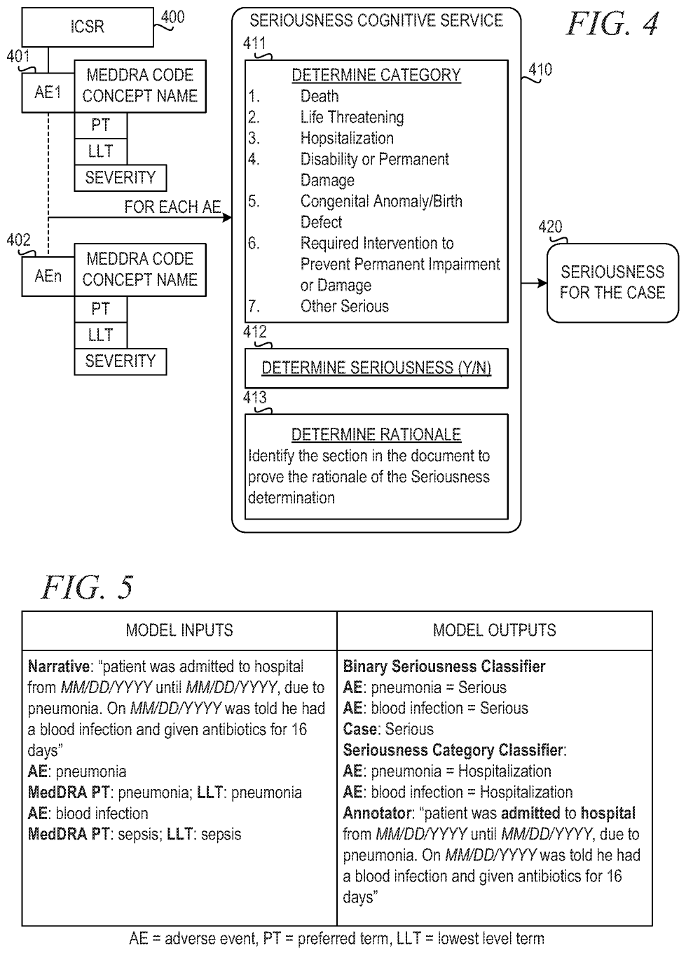

FIG. 4 is a block diagram of a seriousness cognitive service in accordance with an illustrative embodiment. As shown in FIG. 4, the seriousness cognitive service 410 may receive a case 400, such as a Federal Drug Administration (FDA) Individual Case Safety Report (ICSR), and information indicating defined seriousness categories/topics and natural language patterns corresponding to such categories/topics for purposes of cognitive matching using natural language processes. The case 400 comprises a plurality of adverse events (AE1, . . . , AEn) 401, 402 having associated contextual data. For example, in case 400, each AE 401, 402 may include MedDRA code, concept name, preferred term (PT), lower-level temi (LLT), severity, and the like, which may be fed into the seriousness cognitive service 410, which determines whether each AE is a serious event, categorize the AE into one or more seriousness categories, and identifies rationale (e.g., keywords). Seriousness cognitive service 410 aggregates the seriousness results for the plurality of AEs 401, 402 to generate a seriousness for the case 400.

MedDRA or Medical Dictionary for Regulatory Activities is a clinically validated international medical terminology dictionary (and thesaurus) used by regulatory authorities in the pharmaceutical industry during the regulatory process, from pre-marketing (clinical research phase 0 to phase 3) to post-marketing activities (pharmacovigilance or clinical research phase 4), and for safety information data entry, retrieval, evaluation, and presentation. In addition, it is the adverse event classification dictionary endorsed by the International Conference on Harmonisation of Technical Requirements for Registration of Pharmaceuticals for Human Use (ICH).

The analysis of the AEs and the case as a whole may involve top-down (from case to AE) analysis, bottom-up (from AE to case) analysis, structured fields vs. cognitive assessment (reporter seriousness <-> company seriousness, binary (yes/no) vs. N-ary (category classification), etc. Each approach may have different predictions and confidences, which may be fed into a super-classifier (e.g., neural net) to determine case seriousness, weighting different approaches (e.g., structured fields say not serious but other approaches may say there is a reason for this to be considered serious), and the super-classifier determines the seriousness of the case based on a consolidation of these approaches. One methodology may be to indicate seriousness if anything indicates serious in any of the different analysis approaches. Other methodologies may weight the different analyses for different types of seriousness determinations, where these weights may be machine learned through training and user feedback mechanisms.

Seriousness cognitive service 410 evaluates seriousness at the AE granularity. In block 411, the seriousness cognitive service 410 determines, for a given AE 401, 402, seriousness in each seriousness category including the following:

1. Death

2. Life Threatening

3. Hospitalization

4. Disability or Permanent Damage

5. Congenital Anomaly/Birth Defect

6. Required Intervention to Prevent Permanent Impairment or Damage

7. Other Serious Important Medical Events

As an example, the Death category may include the following topics or keywords: deaths, death, cardiac death, sudden death, completed suicide, brain death, fetal death, accidental death, cardiac arrest, cardiac failure, fear of death, etc. The Congenital Anomaly category may include the following topics or keywords: pregnancy, pregnancies, exposure during pregnancy, congenital anomaly, congenital skin disorder, fetal heart rate decreased, swelling face, fetal disorder, maternal exposure before pregnancy, congenital anomalies, etc. The Disability or Permanent Damage category may include the following topics or keywords: memory impairment, nerve injury, visual impairment, physical disability, impaired driving ability, renal impairment, surgery, surgeries, surgical, surgically, angiocdema, etc. The Required Intervention to Prevent Permanent Impairment or Damage category may include the following topics or keywords: procedure, surgical and medical procedures, back surgery, procedural, complication, spinal surgery, endodontic procedure, spinal fusion surgery, surgical failure, obesity surgery, etc. The Life Threatening category may include the following topics or keywords: myocardial infarction, cardiac failure, blood pressure increased, renal infarction, blood potassium increased, respiratory failure, cardiac arrest, blood glucose increased, etc. The Hospitalization category may include the following topics or keywords: hospitals, hospital, in hospital, hospitalized, hemorrhage, multiple injuries, gastric ulcer hemorrhage, coronary artery, stenosis, cerebral hemorrhage, etc.

In block 412, the seriousness cognitive service 410 determines a seriousness result 420 for the overall case 400. And in block 413, the seriousness cognitive service 410 determines a rationale for the seriousness determination. The seriousness cognitive service identifies the section (e.g., keywords) in the document to prove the rationale of the seriousness determination. In one embodiment, blocks 411, 412, 413 are performed in parallel.

FIG. 5 depicts an example of model input and output for the seriousness cognitive service in accordance with an illustrative embodiment. The model inputs include the narrative written into the patient's case. The model inputs also include one or more AEs, which include “pneumonia” and “blood infection” in the example shown in FIG. 5. The model input also includes the MedDRA preferred term (PT) and lower-level term (LLT) associated with each AE.

The model outputs include a binary seriousness classifier for each AE. In the depicted example, the binary seriousness result for the AE of “pneumonia” is “Serious,” and the binary seriousness result for the AE of “blood infection” is “Serious.” The binary seriousness result for the overall case is “Serious.” The model outputs a seriousness category classifier for each AE. In the depicted example, the seriousness category classifier for the AE of “pneumonia” is “Hospitalization,” and the seriousness category classifier for the AE of “blood infection” is “Hospitalization.” The model outputs also include an annotator that highlights terms in the narrative that support a rationale for the finding of seriousness and seriousness categorization.

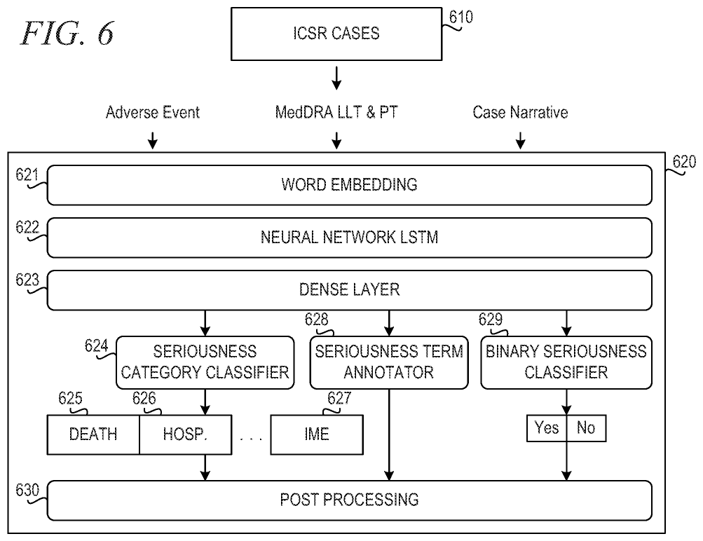

FIG. 6 is a block diagram illustrating a seriousness determination cognitive module for a seriousness cognitive service in accordance with an illustrative embodiment. Seriousness determination cognitive module 620 comprises three neural networks. It determines seriousness of adverse events by a binary adverse event level seriousness classifier, a classifier for determining seriousness categorization at the adverse event level, and an annotator for identifying seriousness criteria terms to provide supporting evidence at the document level.

Seriousness determination cognitive module 620 receives ICSR cases 610, which include at least one adverse event, a MedDRA lower level term (LLT) and preferred term (PT) for the adverse event, and a case narrative. Input to the seriousness determination module 620 can be extended to accept additional input, such as the severity of the events or the like. Input to the seriousness cognitive service can be an output of another cognitive service or could be made available by human practitioners.

Word embedding component 621 comprises a set of language modeling and feature learning techniques in natural language processing (NLP) where words or phrases from the vocabulary are mapped to vectors of real numbers. Conceptually, word embedding component 621 involves a mathematical embedding from a space with many dimensions per word to a continuous vector space with a much lower dimension. Methods to generate this mapping include neural networks, dimensionality reduction on the word co-occurrence matrix, probabilistic models, explainable knowledge base method, and explicit representation in terms of the context in which words appear. Essentially, word embedding component 621 converts a natural language text of words and terms into a vector of numerical values that can be processed by the neural networks. Word and phrase embeddings, when used as the underlying input representation, have been shown to boost the performance in NLP tasks such as syntactic parsing and sentiment analysis.

Neural network long short-term memory (LS™) component 622 is an artificial recurrent neural network (RNN) architecture used in the field of deep learning. Unlike standard feedforward neural networks, LS™ has feedback connections that make it a “general purpose computer.” That is, it can compute anything that a Turing machine can. Neural network LS™ component 622 can not only process single data points, but also entire sequences of data. A common LS™ unit is composed of a cell, an input gate, an output gate, and a forget gate. The cell remembers values over arbitrary time intervals and the three gates regulate the flow of information into and out of the cell. LS™ networks are well-suited to classifying, processing, and making predictions based on time series data, since there can be lags of unknown duration between important events in a time series. LSTMs were developed to deal with the exploding and vanishing gradient problems that can be encountered when training traditional RNNs. Relative insensitivity to gap length is an advantage of LS™ over RNNs, hidden Markov models and other sequence learning methods in numerous applications.

Dense layer component 623 is a classic fully connected neural network layer: each input node is connected to each output node. The output nodes provide inputs to seriousness category classifier 624, seriousness term annotator 628, and binary seriousness classifier 629.

Seriousness category classifier 624 provides an output for each seriousness category (e.g., Death 625, Hospitalization 626, Other Serious Important Medical Events (IME) 627, etc.). Thus, Seriousness category classifier 624 outputs a plurality of binary determinations, one for each seriousness category.

Seriousness term annotator 628 highlights terms in the case narrative that provide a rationale for the seriousness determination. These terms may be based on the topics or keywords, such as those described in the examples above, with regard to each seriousness category. Binary seriousness classifier 629 provides a yes/no determination for the overall seriousness for the patient's case.

Post processing component 630 combines the outputs of the seriousness category classifier 624, the seriousness term annotator 628, and the binary seriousness classifier 629. Therefore, post processing component 630 provides a binary seriousness determination for each adverse event, a seriousness category for each adverse event determined to be serious, and an annotated case narrative providing a rationale for the seriousness determination.

As will be described below, an expectedness cognitive service receives an ICSR case as input, which comprises one or more adverse events (AE1, . . . , AEn) having associated contextual data including, for example, a suspect drug, seriousness, severity, MedDRA code, concept name, preferred term (PT), lower level term (LLT), etc. For each adverse event, the expectedness cognitive service evaluates the adverse event in accordance with a plurality of different drug label service repositories indicating expected side effects of drugs. The expectedness cognitive service operates on a tuple granularity, where the tuple is expectedness values for the plurality of different conventions for specifying expected side effects of drugs (e.g., Investigator's Brochure (IB), Summary of Product Characteristics (SMPC), Company Core Data Sheet (CCDS), United States Prescribing Information (USPI), etc.). This repository is the drug company issued documents, which are updated periodically and comprise drug label information (e.g., “if taken on an empty stomach can cause nausea”).

In addition, the expectedness cognitive service may cognitively process the context of an adverse event indication in the case to determine whether the drug label information, as specified in the repository, applies to the context in which the adverse event was identified. For example, while nausea may be indicated as a side effect if the drug is taken on an empty stomach, there may be other instances where nausea is not expected, yet may be indicated in the case. The expectedness cognitive service looks at the context of the nausea to determine whether the adverse event is expected or not and outputs an indication of expectedness.

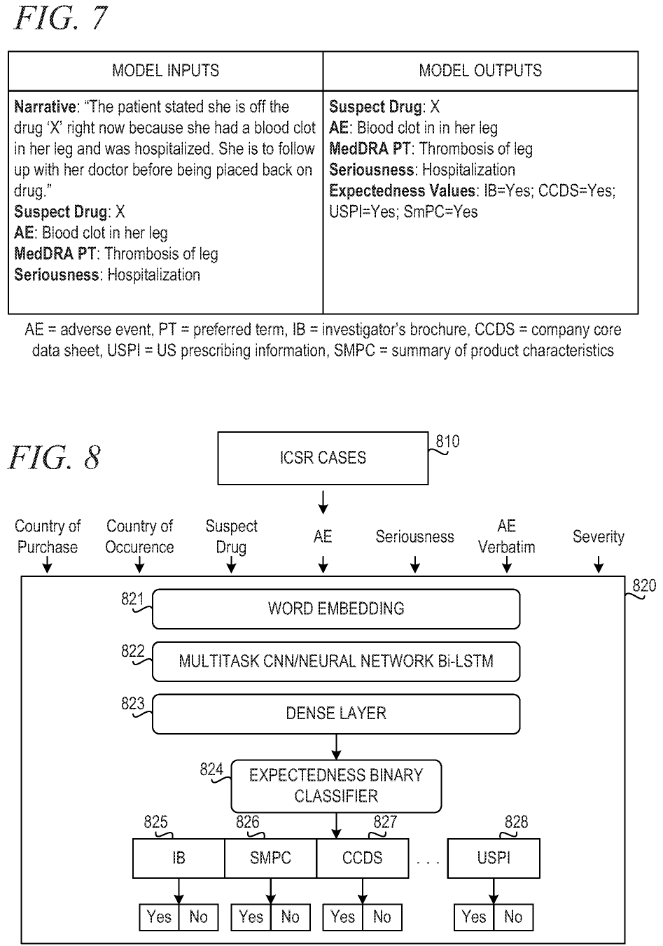

FIG. 7 depicts an example of model input and output for the expectedness cognitive service in accordance with an illustrative embodiment. The model inputs include the narrative written into the patient's case. The model inputs also include a suspect drug and one or more AEs, which includes “blood clot in her leg” in the example shown in FIG. 7. The model input also includes the MedDRA preferred term (PT) and seriousness associated with each AE. In one example embodiment, the seriousness may be the seriousness determined by the seriousness cognitive service described above. In an alternative embodiment, the seriousness may be provided by another cognitive service or by a human expert.

The model outputs include the suspect drug, the adverse event, the MedDRA preferred term (PT), the seriousness, and expectedness values for a plurality of drug label service repositories. In the depicted example, the expectedness cognitive service determines that for a given suspect drug (X), the adverse event of “Blood clot in her leg” was expected when considered with respect to Investigator's Brochure (IB), Company Core Data Sheet (CCDS), United States Prescribing Information (USPI), and Summary of Product Characteristics (SMPC).

FIG. 8 is a block diagram illustrating an expectedness determination cognitive module for an expectedness cognitive service in accordance with an illustrative embodiment. Expectedness determination cognitive module 820 receives as input ICSR cases 810, which comprise a suspect drug, an adverse event, a country of purchase of the suspect drug, a country of occurrence of the adverse event, a seriousness, the adverse event verbatim, and a severity. Other inputs may include, for example, date of purchase of the suspect drug, date of occurrence of the adverse event, or the like. In one example embodiment, the seriousness may be the seriousness determined by the seriousness cognitive service described above. In an alternative embodiment, the seriousness may be provided by another cognitive service or by a human expert.

Word embedding component 821 comprises a set of language modeling and feature learning techniques in natural language processing (NLP) where words or phrases from the vocabulary are mapped to vectors of real numbers. Conceptually, word embedding component 821 involves a mathematical embedding from a space with many dimensions per word to a continuous vector space with a much lower dimension. Methods to generate this mapping include neural networks, dimensionality reduction on the word co-occurrence matrix, probabilistic models, explainable knowledge base method, and explicit representation in terms of the context in which words appear. Essentially, word embedding component 821 converts a natural language text of words and terms into a vector of numerical values that can be processed by the neural networks.

Multitask convolutional neural network (CNN) or neural network bidirectional long short-term memory (Bi-LS™) component 822 is a multitask CNN or bidirectional recurrent neural network. CNNs are regularized versions of multilayer perceptrons. Multilayer perceptrons usually refer to fully connected networks, that is, each neuron in one layer is connected to all neurons in the next layer. The “fully-connectedness” of these networks makes them prone to overitting data. CNNs take advantage of the hierarchical pattern in data and assemble more complex patterns using smaller and simpler patterns. Therefore, on the scale of connectedness and complexity, CNNs are on the lower extreme. Bidirectional Recurrent Neural Networks (BRNN) connect two hidden layers of opposite directions to the same output. With this form of generative deep learning, the output layer can get information from past (backwards) and future (forward) states simultaneously. BRNNs were introduced to increase the amount of input information available to the network. BRNNs are especially useful when the context of the input is needed.

Dense layer component 823 is a classic fully connected neural network layer: each input node is connected to each output node. The output nodes provide inputs to expectedness classifier 824.

Expectedness classifier 824 provides an expectedness value for each of a plurality of drug label service repositories. In the example depicted in FIG. 8, the repositories include Investigator's Brochure (IB) 825, Summary of Product Characteristics (SMPC) 826, Company Core Data Sheet (CCDS) 827, and United States Prescribing Information (USPI) 828. Each of the values 825-828 represents a binary determination (yes/no) of whether the adverse event is expected for the suspect drug. The repositories may include any combination of one or more of the repositories 825-828 shown in FIG. 8 as well as other available drug labeling document repositories.

For each combination of suspect drug, adverse event, seriousness, country of purchase, country of adverse event occurrence, date of purchase, date of adverse event occurrence, etc., the expectedness cognitive service determines expectedness (yes/no) based on the drug Company Core Data Sheet (CCDS). The CCDS lookup should consider worldwide CCDS, country override, time version of the CCDS to determine the expectedness. In one example embodiment, the expectedness cognitive service determines expectedness across all geographies where the drug is available (based on CCDS repository) irrespective of the country of occurrence.

In one embodiment, the expectedness cognitive service determines the expectedness automatically, or, alternatively, expects severity to be available as an input along with each adverse event. A cognitive determination of severity may be performed to provide better expectedness classification.

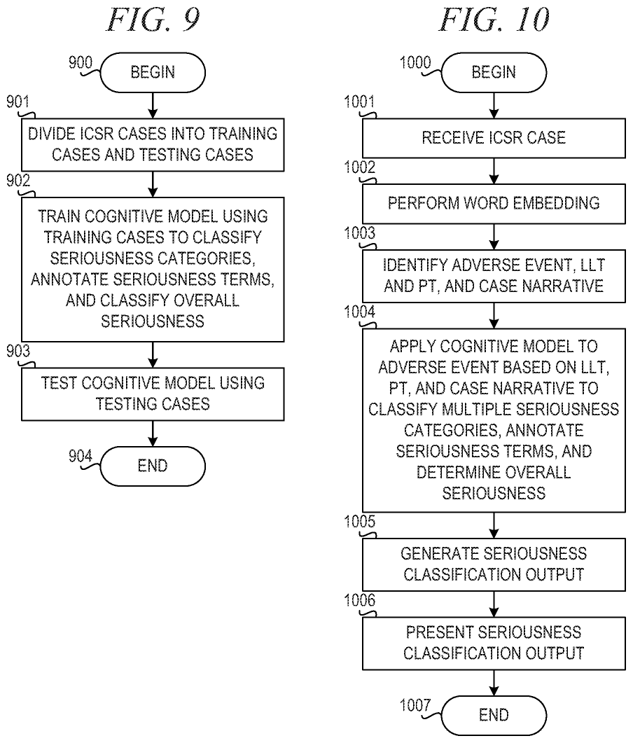

FIG. 9 is a flowchart illustrating operation of a mechanism for training a seriousness cognitive service model in accordance with an illustrative embodiment. Operation begins (block 900), and the mechanism divides ICSR cases into training cases and testing cases (block 901). The mechanism trains the cognitive model using the training cases to classify seriousness categories, annotate seriousness terms, and classify overall seriousness for adverse events in each ICSR case (block 902). The mechanism then tests the cognitive model using the testing cases (block 903). Thereafter, operation ends (block 904).

FIG. 10 is a flowchart illustrating operation of a seriousness cognitive service in accordance with an illustrative embodiment. Operation begins (block 1000), and the seriousness cognitive service receives an ICSR case (block 1001). The seriousness cognitive service performs word embedding (block 1002) and identifies an adverse event, lower level term (LLT), preferred term (PT), and case narrative in the ICSR case (bock 1003). The seriousness cognitive service then applies the cognitive model to the adverse event based on the LLT, PT, and case narrative to classify multiple seriousness categories, annotate the seriousness terms in the case narrative, and determine an overall seriousness for the ICSR case (block 1004). In one embodiment, the seriousness cognitive service may perform a seriousness classification for multiple adverse events by iterating using the same ICSR for each adverse event.

Then, the seriousness cognitive service generates a seriousness classification output for the ICSR case (block 1005) and presents the seriousness classification output to a user (block 1006). Thereafter, operation ends (block 1007). In one embodiment, the seriousness cognitive service may provide the seriousness classification output to another cognitive service, such as an expectedness cognitive service, or to a healthcare cognitive decision support system to aid in generating a cognitive summary of a patient's case.

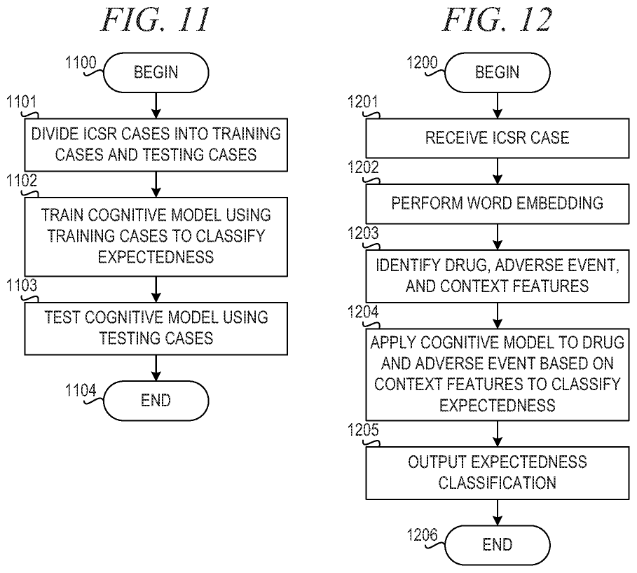

FIG. 11 is a flowchart illustrating operation of a mechanism for training an expectedness cognitive service model in accordance with an illustrative embodiment. Operation begins (block 1100), and the mechanism divides ICSR cases into training cases and testing cases (block 1101). The mechanism trains a cognitive model using the training cases to classify expectedness of adverse events with respect to suspect drugs (block 1102). The mechanism then tests the cognitive model using the testing cases (block 1103). Thereafter, operation ends (block 1104).

FIG. 12 is a flowchart illustrating operation of an expectedness cognitive service in accordance with an illustrative embodiment. Operation begins (block 1200), and the expectedness cognitive service receives an ICSR case (block 1201). The expectedness cognitive service performs word embedding (block 1202) and identifies a suspect drug, an adverse event, and context features (block 1203). In one example embodiment, the context features may include country of purchase of the suspect drug, date of purchase of the suspect drug, country of occurrence of the adverse event, date of occurrence of the adverse event, seriousness, and severity.

The expectedness cognitive service then applies the cognitive model to the suspect drug and adverse event based on the context features to classify expectedness (block 1204). In accordance with an illustrative embodiment, the expectedness cognitive service classifies expectedness with respect to a plurality of drug label service repositories, thus providing a plurality of binary expectedness values, one for each repository. In one embodiment, the expectedness cognitive service classifies expectedness for a plurality of adverse events in the ICSR by iterating using the same ICSR for each adverse event in the ICSR case. The expectedness cognitive service outputs the expectedness classification (block 1205), and operation ends (block 1206). In one embodiment, the seriousness cognitive service may provide the expectedness classification output to another cognitive service or to a healthcare cognitive decision support system to aid in generating a cognitive summary of a patient's case.

In one example embodiment, the expectedness cognitive service may determine whether a given drug label service repository needs to be updated based on whether a suspect drug is strongly correlated with a particular adverse event. For example, if the result for one repository consistently contradicts the other repository for a given combination of a suspect drug and adverse event, then the expectedness cognitive service may inform an administrator or computer system associated with that repository about the possible side effect for the suspect drug. Similarly, if a given repository lists a particular adverse event as a side effect for a suspect drug but the expectedness cognitive service consistently provides a negative value for that repository, then the expectedness cognitive service may inform an administrator or computer system associated with the given repository that the adverse event may not be a side effect for the suspect drug.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising a processor and a memory, the memory comprising instructions that are executed by the processor to specifically configure the processor to implement an expectedness cognitive service for identifying expectedness of a patient case with respect to a suspect drug, the method comprising:
receiving, by the expectedness cognitive service executing in the data processing system, a patient case;
identifying, by the expectedness cognitive service, a suspect drug, an adverse event, and context features based on the patient case;
determining, by an expectedness binary classifier within the expectedness cognitive service, a plurality of expectedness classifications for the adverse event with respect to a plurality of drug labeling service repositories; and
generating and outputting, by the expectedness cognitive service, an expectedness classification output comprising the plurality of expectedness classifications.

2. The method of claim 1, wherein the expectedness cognitive service comprises a word embedding component, a neural network component, and a dense layer component for providing a combination of weighted outputs from the neural network to the expectedness binary classifier.

3. The method of claim 2, wherein the neural network component comprises a multitask convolutional neural network.

4. The method of claim 2, wherein the neural network component comprises a bidirectional long short-term memory (LSTM) neural network.

5. The method of claim 1, wherein the plurality of drug labeling service repositories comprise Investigator's Brochure (IB), Summary of Product Characteristics (SMPC), Company Core Data Sheet (CCDS), or United States Prescribing Information (USPI).

6. The method of claim 1, wherein the context features comprise country of purchase of the suspect drug, date of purchase of the suspect drug, country of occurrence of the adverse event, date of occurrence of the adverse event, seriousness of the adverse event, or severity of the adverse event.

7. The method of claim 1, wherein determining the plurality of expectedness classifications comprises providing the suspect drug, the adverse event, and the context features as inputs to a cognitive model.

8. The method of claim 7, wherein the cognitive model comprises a neural network.

9. A computer program product comprising a computer readable storage medium having a computer readable program stored therein, wherein the computer readable program comprises instructions, which when executed on a processor of a computing device causes the computing device to implement an expectedness cognitive service for identifying expectedness of a patient case with respect to a suspect drug, wherein the computer readable program causes the computing device to:
receiving, by the expectedness cognitive service executing in the data processing system, a patient case;
identifying, by the expectedness cognitive service, a suspect drug, an adverse event, and context features based on the patient case;
determining, by an expectedness binary classifier within the expectedness cognitive service, a plurality of expectedness classifications for the adverse event with respect to a plurality of drug labeling service repositories; and
generating and outputting, by the expectedness cognitive service, an expectedness classification output comprising the plurality of expectedness classifications.

10. The computer program product of claim 9, wherein the expectedness cognitive service comprises a word embedding component, a neural network component, and a dense layer component for providing a combination of weighted outputs from the neural network to the expectedness binary classifier.

11. The computer program product of claim 10, wherein the neural network component comprises a multitask convolutional neural network.

12. The computer program product of claim 10, wherein the neural network component comprises a bidirectional long short-term memory (LSTM) neural network.

13. The computer program product of claim 9, wherein the plurality of drug labeling service repositories comprise Investigator's Brochure (IB), Summary of Product Characteristics (SMPC), Company Core Data Sheet (CCDS), or United States Prescribing Information (USPI).

14. The computer program product of claim 9, wherein the context features comprise country of purchase of the suspect drug, date of purchase of the suspect drug, country of occurrence of the adverse event, date of occurrence of the adverse event, seriousness of the adverse event, or severity of the adverse event.

15. The computer program product of claim 9, wherein determining the plurality of expectedness classifications comprises providing the suspect drug, the adverse event, and the context features as inputs to a cognitive model.

16. The computer program product of claim 15, wherein the cognitive model comprises a neural network.

17. A computing device comprising:
a processor; and
a memory coupled to the processor, wherein the memory comprises instructions, which when executed on a processor of a computing device causes the computing device to implement an expectedness cognitive service for identifying expectedness of a patient case with respect to a suspect drug, wherein the instructions cause the processor to:
receiving, by the expectedness cognitive service executing in the data processing system, a patient case;
identifying, by the expectedness cognitive service, a suspect drug, an adverse event, and context features based on the patient case;
determining, by an expectedness binary classifier within the expectedness cognitive service, a plurality of expectedness classifications for the adverse event with respect to a plurality of drug labeling service repositories; and
generating and outputting, by the expectedness cognitive service, an expectedness classification output comprising the plurality of expectedness classifications.

18. The computing device of claim 17, wherein the expectedness cognitive service comprises a word embedding component, a neural network component, and a dense layer component for providing a combination of weighted outputs from the neural network to the expectedness binary classifier.

19. The computing device of claim 17, wherein the plurality of drug labeling service repositories comprise Investigator's Brochure (IB), Summary of Product Characteristics (SMPC), Company Core Data Sheet (CCDS), or United States Prescribing Information (USPI).

20. The computing device of claim 17, wherein the context features comprise country of purchase of the suspect drug, date of purchase of the suspect drug, country of occurrence of the adverse event, date of occurrence of the adverse event, seriousness of the adverse event, or severity of the adverse event.
